(19) United States
(12) Patent Application Publication
(10) Pub. No.: US 2019/0074081 A1
(43) Pub. Date: Mar. 7, 2019

(54) Digital Behavioral Health Platform

(71) Applicants: Caroline J. Easton, Pittsford, NY (US); Richard Doolittle, Pittsford, NY (US)

(72) Inventors: Caroline J. Easton, Pittsford, NY (US); Richard Doolittle, Pittsford, NY (US)

(73) Assignee: Rochester Institute of Technology, Rochester, NY (US)

(21) Appl. No.: 16/117,713

(22) Filed: Aug. 30, 2018

Related U.S. Application Data
(60) Provisional application No. 62/553,389, filed on Sep. 1, 2017.

(51) Int. Cl. G16H 20/70; G16H 10/60; G06F 9/451

(52) U.S. Cl. CPC G16H 20/70; G06F 9/453; G16H 10/60

(57) ABSTRACT

A platform and method for administering a digital behavioral health treatment to an individual, includes customizing and personalizing a digital avatar health coach and an interactive therapy platform of evidenced based cognitive behavioral therapy which targets and treats maladaptive behaviors in the individual to improve treatment outcomes, wherein the digital platform is standardized and specific to target specific maladaptive behaviors and implements positive, healthier, and prosocial behaviors incorporating the mechanisms of social learning theory, classical conditioning, and operant conditioning.

### CROSS REFERENCE

This application claims the benefit of the filing date of U.S. Provisional Patent Application Ser. No. 62/553,389, filed Sep. 1, 2017, which is hereby incorporated by reference in its entirety.

### FIELD

The disclosure relates to a platform and method for administering digital cognitive behavioral health treatment, and in particular to digital cognitive behavioral health treatment for correcting maladaptive behaviors.

### BACKGROUND

Research shows that clients who attempt to practice their Cognitive Behavioral Therapy (CBT) coping skill exercises have significantly better treatment outcomes (“Practice Makes Progress” by Carroll-NIH Overview Study)

The problem was resolved in the past by assigning paper and pencil coping skills exercises and using human therapist and coaches. The therapists have to be trained to be competent and adhere to the model of care. However, if therapists do not adhere, the specific coping skill may not be adhered to.

There is now a CBT4CBT computerized version of CBT for clients with Addictive Disorders. However, this does not involve any digital or interactive take home coping skills exercises nor does it provide an Avatar coach who can be present to show and model the specific coping skills and health behaviors. The CBT4CBT approach targets only addiction. It uses humans. It uses older forms of multimedia such as videotaped examples, vignettes, movies, verbal instructions from humans and voiceovers. Although CBT4CBT has some interactive practice exercises. It doesn't have a digital interactive CBT coping skills exercise for patients to practice on their smart phones, Ipads or across a number of inexpensive digital platforms (e.g., fire kindle).

### SUMMARY

In accordance with one aspect of the present disclosure, there is provided a method for administering a digital behavioral health platform to an individual, including: customizing a digital avatar health coach; practicing a set of coping skills provided by an interactive therapy platform of evidenced based cognitive behavioral therapy which targets and treats maladaptive behaviors in the individual to improve treatment outcomes; and interacting with the avatar health coach to provide personalized feedback, wherein the digital platform is standardized and specific to target specific maladaptive behaviors and implements positive, healthier, and prosocial behaviors which incorporates the mechanisms of social learning theory, classical conditioning, and operant conditioning based on the feedback.

In accordance with another aspect of the present disclosure, there is provided a digital behavioral health platform for an individual, including: a customizable digital avatar health coach; and an interactive therapy platform of evidenced based cognitive behavioral therapy which targets and treats maladaptive behaviors in the individual to improve treatment outcomes based on the feedback from practicing a set of coping skills.

These and other aspects of the present disclosure will become apparent upon a review of the following detailed description and the claims appended thereto.

### BRIEF DESCRIPTION OF THE DRAWINGS

FIG. 1 represents a flow chart showing the administrator's role in the health platform in accordance with an embodiment of the present disclosure;

FIGS. 2-4 represent a flow chart showing the patient's role in the health platform in accordance with an embodiment of the present disclosure;

FIG. 5 represents a flow chart showing the doctor's role in the health platform in accordance with an embodiment of the present disclosure;

FIG. 6 is a screen shot of the Avatar asking and assessing the user's relationship between conflict and substance use in accordance with Example 1;

FIG. 7 is a screen shot of the Avatar to be customized to hair style, clothing and skin color in an office background in accordance with Example 1;

FIG. 8 is a screen shot of a menu of options of 10 of 12 topics of skill sets to be assigned in accordance with Example 1;

FIG. 9 is a screen shot of some assessment questions for anger in accordance with Example 1; and

FIG. 10 is a screen shot of achievements earned in accordance with Example 1.

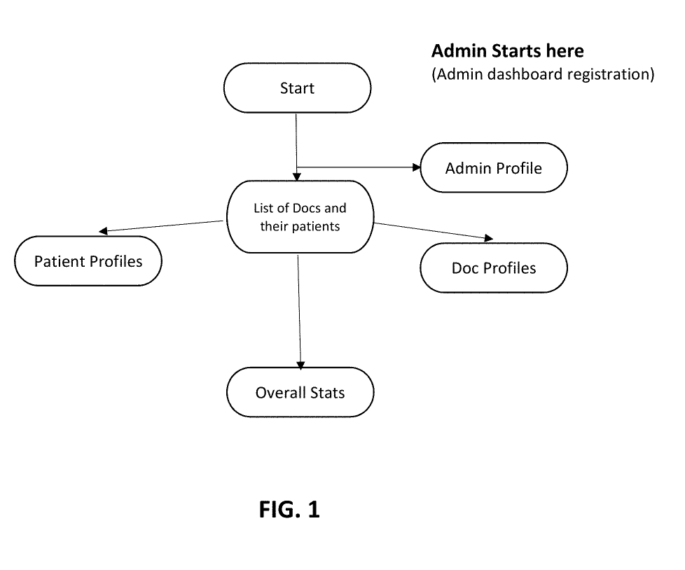

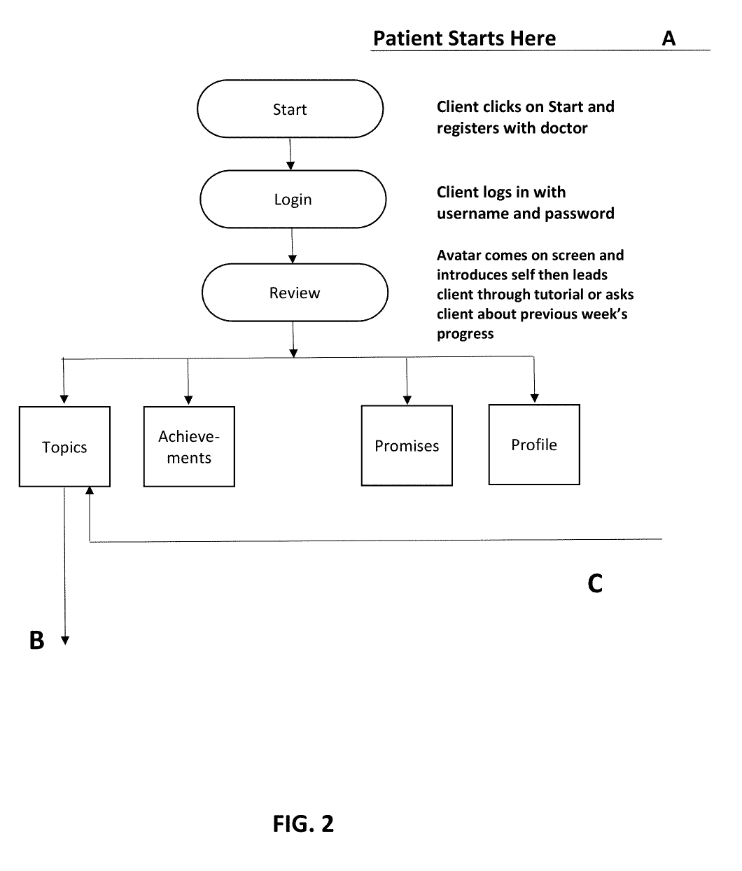

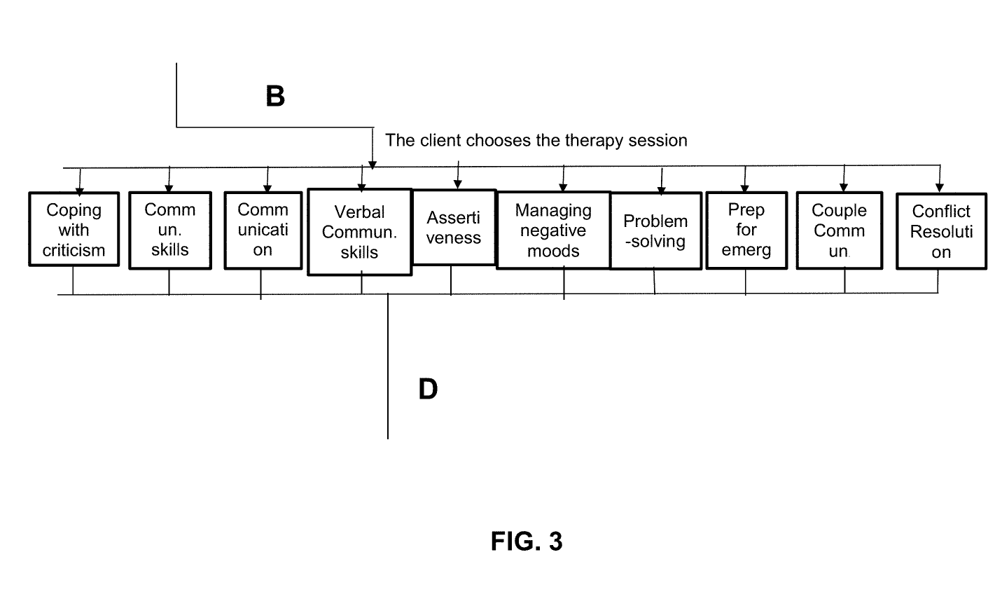

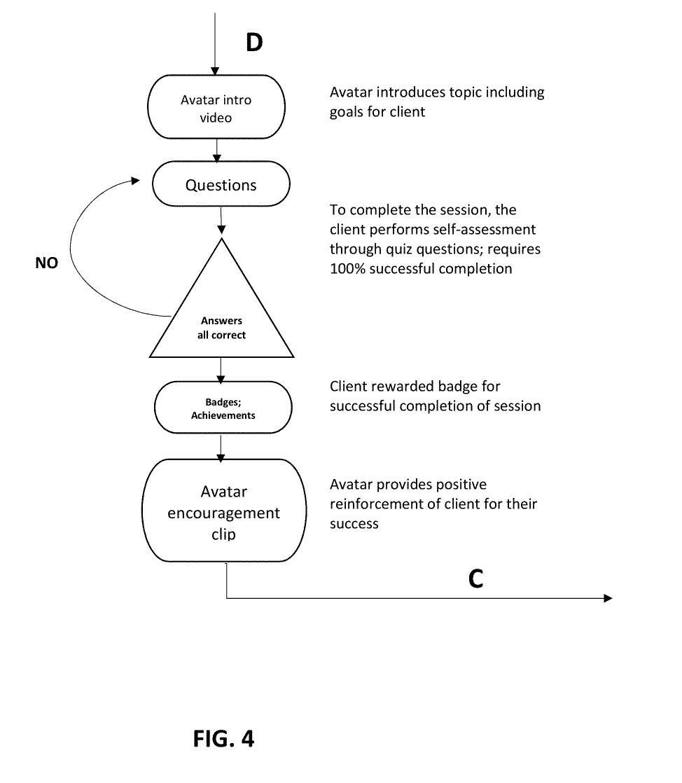

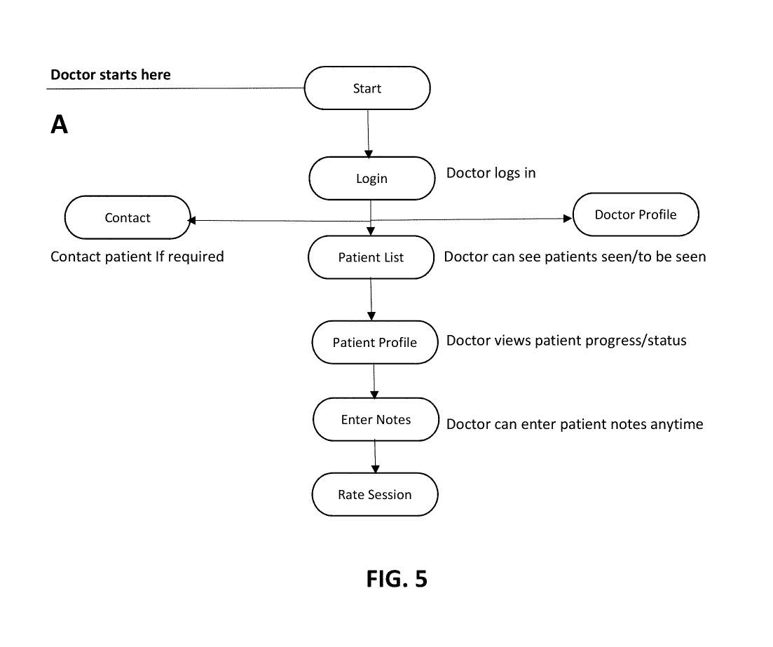

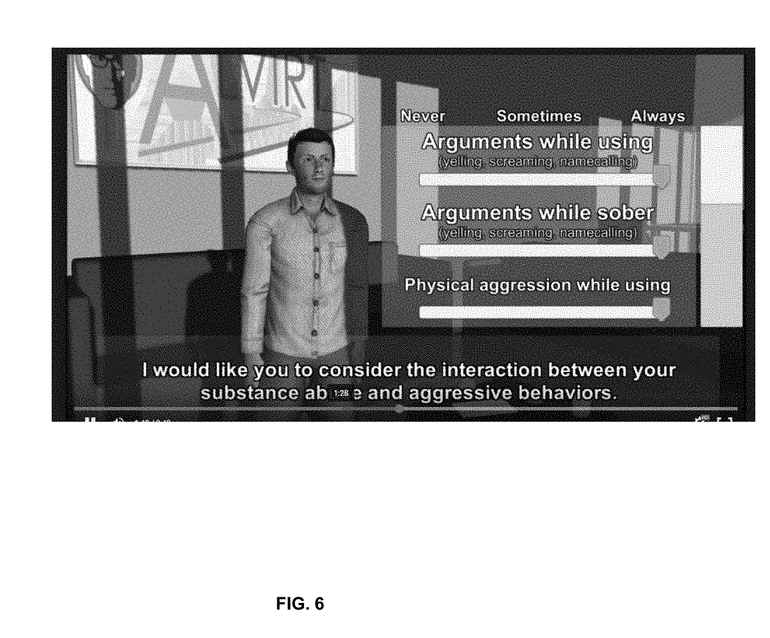

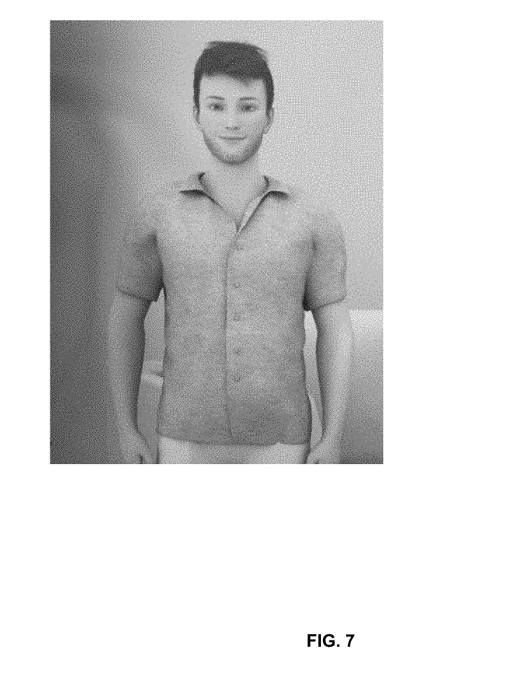

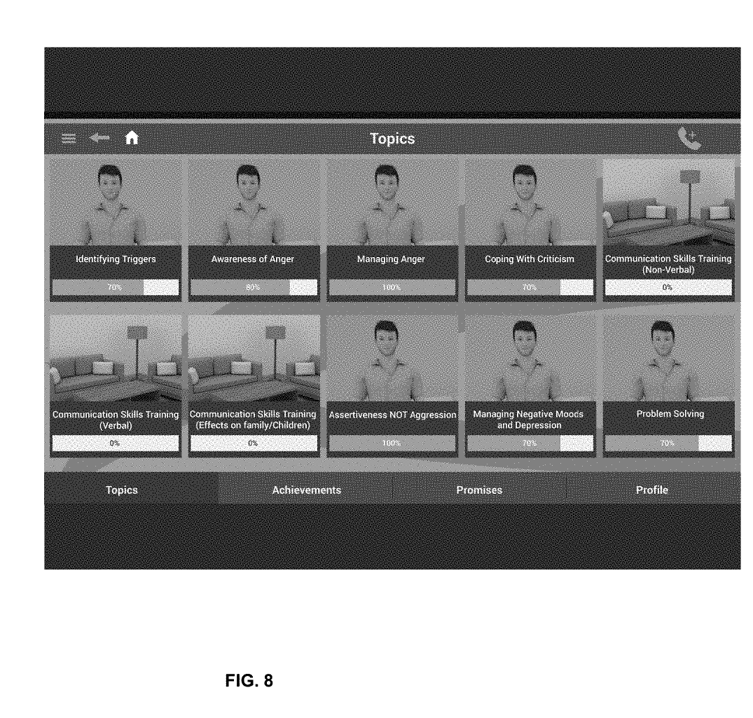

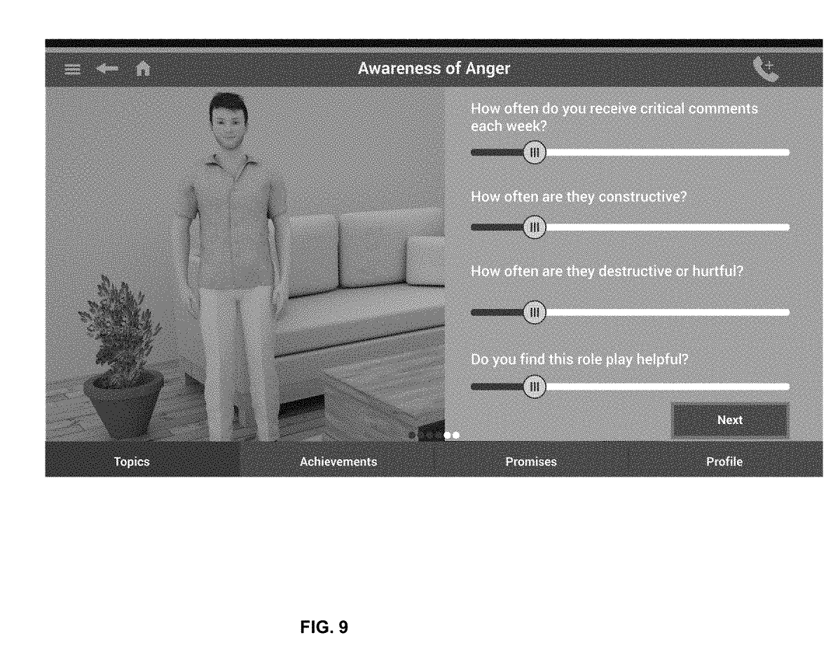

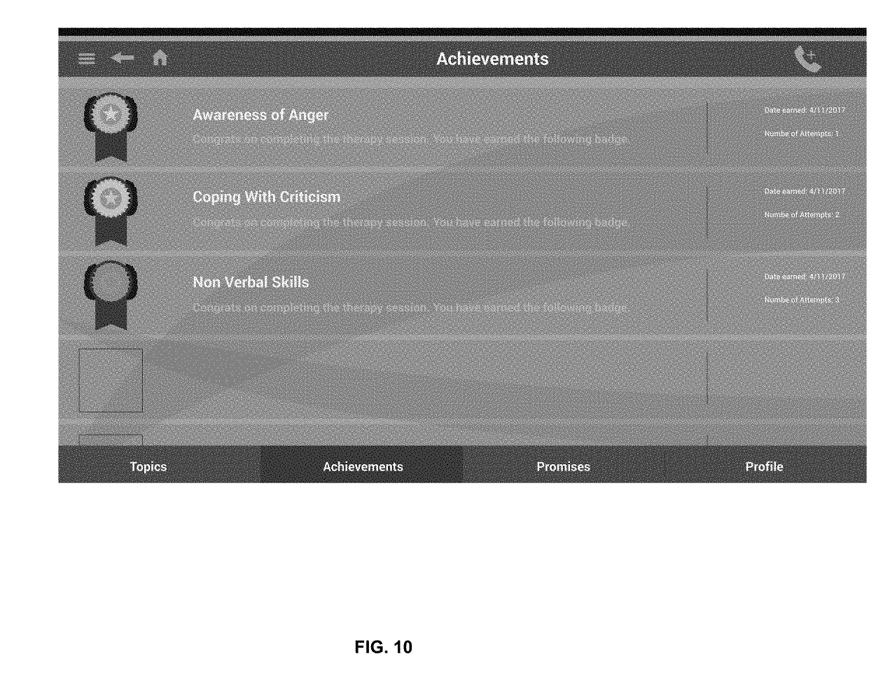

### DETAILED DESCRIPTION

In an embodiment, of a multi-step therapeutic approach toward correcting maladapted behaviors, the therapist introduces clients to a set of behavioral health learning objectives via an interactive and digitized platform: a set of Cognitive Behavioral Therapy topics around the practice of changing behavior in a deliberate fashion. This may take many weeks to achieve. This digitized Cognitive Behavioral Therapy (dCBT) platform is a customizable, interactive behavioral therapy that is client-centered and provides the user with self-assessment in real time. This invention has built in rewards (verbal incentives), positive sounds and rewards (e.g., visual ribbons, trophies and plaques for achievements ascertained in the program). Based on the user's disclosure, the program creates a set of coping skills that is provided to the user to practice. To help the user attain healthier behavior change, the user creates his/her own Avatar that acts as their behavioral therapy coach. The user chooses the race and gender they prefer to have as their therapy coach, a function that is often not available in real world clinical settings because of limited resources and lack of diversity in their human therapists.

Customizable means personalized to the user with respect to gender, ethnicity/racial (Hispanic, African American, Caucasian, Mixed, Other), skin color and overall appearance, e.g., hair style, clothing options and environment for interaction, e.g., therapist office or nature setting. For example, The Avatar asks the user (John) to personalize their Coach by choosing gender (male, female, non-gender specific option), ethnicity/racial option (choose skin colors—light complexion, light brown, brown, dark brown, very dark brown), hair style (long hair, short hair, short spikey hair, brush cut, short curly hair), clothing options (female khaki slacks, female dress jeans, female dress shirt, female casual shirt, dress with and without scarf, male khaki slacks, male dress jeans, male dress shirt, male casual shirt, male dress shirt with or without a tie, non-gender specific khaki slacks, dress jeans, dress shirt, casual shirt, dress, scarves, ties) and environment (Avatar Coach w/An Office Background or Avatar Coach with Nature Scene in background).

Interactive means a personalized dynamic approach that incorporates the user's answers into an assessment that occurs with each interaction. Interactive involves real time observations of the user's responses. In real time, the user's responses are linked to a decision making tree that is continuously re-evaluating progress. Based on key words and responses, new tasks and skill sets are assigned to the user to practice and reassess. The most relevant skills are assigned based on the user's current response, current problems and strategies/skill sets needed & most likely to be used. Each skill assigned is re-assessed and linked to the user's progress and achievements. The user is always rating their likelihood of using the assigned skill set, their amount of practice with a skill set, their satisfaction with the skill set, how helpful the skill set was, how knowledgeable they are about the skill set and whether they are improving or now with each skill or combination of skills. Each interaction incorporates and integrates past and current performance into a decision making tree. In the real time, progress gets assessed and if progress is not made with the most common skill sets assigned, new skill sets are assigned to help the user improve. The most useful skill sets linked to the user's progress are stored and re-integrated into the treatment plan when key progress has been linked to a particular skill set assigned. This dynamic approach uses artificial intelligence to help make multiple recommendations of possible treatments (e.g., skill sets) linked to multiple time points, using expert systems (e.g., cognitive computing) for clinical applications such as behavioral therapies (CBT) Interactive that utilizes artificial intelligence is defined as having an expert knowledge base of skill sets. The platform uses algorithms to suggest appropriate coping skills and practice exercises based on expert system models (e.g., published evidenced based practices shown to be effective. In this case, CBT is used to target specific maladaptive behaviors). The algorithms are based on public domain information and can be derived by persons of ordinary skill in the art. Although past practices are dependent on expert opinions and statistical averages, the current platform is more individualized and the program and algorithm constantly re-assess'what is most useful and what skill sets have been linked to the best progress for each user. It keeps track of progress from day 1 of the interaction for each interaction. Each assessment and progress (behavior improvement) is tracked. The platform learns from each interaction and each progress noted (or not observed). That is, the platform learns from the user's clinical data presented in the assessment, achievements and progress. The program assess' patterns of improvements. By incorporating assessments with each interaction, the system develops new knowledge and modifies existing data/knowledge within the evidenced based model to fit a real world situation in real time that is individualized to the user.

The Avatar therapy coach, a client centered, customizable feature of this platform, provides verbal incentives, rewards and affirmations depending on how the user interacts with the CBT therapy platform. For example, the user is rewarded for interacting with the digitized CBT platform from the moment they log into the program and design their Avatar coach. They experience continuous verbal incentives, verbal rewards, and affirmations all based on elements of operant conditioning to help shape healthy behavior change. Users have opportunities for 24/7 self-assessment and limitless time to practice healthy coping skills with the Avatar's positive affirmations. The platform also uses aspects of social learning theory to show the user how to interact with the digitized CBT platform and how to practice their exercises. The Avatar is a healthy role model who assists the user through the practice of behavior change coping skills.

Once the user discloses their maladaptive behaviors as part of the initial platform, a functional analysis, the coping skills sets necessary for problem behaviors are assigned to the user via an algorithm. The first ⅓ of the digitized therapy session is spent in assessment of the previous week's frequency and severity of the problem behavior and the degree to which the coping skills were practiced. Based on the user's feedback, the program will either ignore negative behaviors or reward positive, prosocial steps at behavior change. The second ⅓ of the digitized therapy session introduces and shows the user the new coping skill to practice in a genuine, empathic and non-confrontational manner. The last ⅓ of the therapy session involves the Avatar behavioral therapy coach showing the user how to do the healthy coping skills. The Avatar then asks the user to practice it at that moment in time. The user chooses the coping skill sets to practice from a menu of options that ask them which coping skill is a skill they are most likely to achieve. As the user interacts, the user is rewarded with pleasant scenery, pleasant sounds and verbal affirmations.

In an embodiment, via the built in slider scales, the user is asked to swipe the slider scales and rate on a likert scale how helpful and satisfied they were with the practice skill set. The user is also asked to rate how likely they are to practice this skill set each day and they are rewarded for any response indicating they will practice.

The user is then given an interactive quiz on the learned skill sets to ascertain whether the user learned the skill set. Once the user achieves a 100% score, the program provides certificates, trophies, ribbons as rewards that are in combination with pleasant sounds and pleasant colors. The user also receives notifications reminding them of their positive steps and awards within the platform. The entire time the user is interacting, an intermittent schedule of partial rewards is occurring to offer the user a chance to earn other badges, certificates or trophies. This is to help avoid habituation to the rewards by offering the intermittent partial reward schedule built into the platform.

Each day and week, the user can see progress of their healthy behavior change and their maladaptive behaviors via a graph to see whether healthy behaviors are increasing, staying the same or decreasing. The user's doctor/therapist can also see the user's notifications of interactions, awards and a bar chart showing whether the maladaptive behavior is changing. Risky behaviors that are endorsed by the user/client are flagged and the clinician/doctor receives an “alert notification” at any point in time to allow for additional supports and treatment necessary to help reduce risky behaviors (e.g., higher level of care, more in-person therapy sessions, additional coping skill sets).

This kind of continuous behavioral modification assessment is only possible through this interactive application. Steps through the app:

a) the client will log-in through their established username and password;

b) Avatar behavioral coach comes on the screen and introduces him/herself and then leads the first-time user client through an introductory CBT tutorial;

c) through this client-centered platform, the client will personalize their Avatar coach with respect to gender, ethnicity/racial options and overall appearance (hair style, clothing options and environment (office or nature setting) as a means of connection and emotional engagement; and

d) the Avatar behavioral coach introduces the client to the series of topics/sessions to be completed as part of the therapeutic protocol. Each session provides the user with:

1) initial and daily assessments of the maladaptive behavior (averaging across days for a weekly score);

2) information about healthier skill sets (e.g., based on the users responses to the daily assessments);

3) a means to practice the skill sets using the app (e.g., practicing anywhere across mobile platforms),

4) the user/client would have multiple topics to cover, typically one topic per week across many weeks. Each topic has numerous coping skill exercises. Clients can interact daily, multiple times in one day, weekly across the therapeutic regimen. Clients/user can continue to interact, practice and repeat topics;

5) a way to assess their interaction satisfaction (e.g., user gives a review and satisfaction of the tools);

6) quiz to learn and reflect on skill acquisition; and

7) the user/client receives notifications to interact if they have not interacted midweek.

In subsequent weeks and in compliance with intended behavioral health outcomes and associated skill sets (as determined by the therapist and the specific maladapted behavior) the Avatar behavioral health coach provides input and positive reinforcement as data on user activity and progress are sent immediately to the therapist through a dedicated, secure, confidential server. Specifically:

A) Once the client/user reviews details of the topic of the intended behavioral change, the Avatar behavioral coach poses a series of questions; all of which need to be answered correctly for the client to complete the session.

B) Once the session is completed, the client is rewarded via an intermittent positive reinforcement schedule so the user does not habituate to the rewards. Rewards include sounds, visual colors, ribbons, certificates of achievement, trophies and verbal affirmations by the Avatar coach. The user is also shown a bar chart showing their positive progress from each interaction each week. The rewards are immediate and a means of positive reinforcement of the user's/client's understanding of the significance of behavior change.

C) Each week, the client will choose to complete the assessments with recommended coping skill exercises depending on how they answer the triggers. The topics are suggested but the user can choose from a menu of options to practice any number of coping skills to complete (even repeat a topic if need be) and go through the same process of review and assessment of their compliance with designed learning outcomes.

Assigned therapists will have routine access to data submitted by clients through each of the topics/exercises. Therapists will:

1) Log in to the app with username and password;

2) Access a list of their assigned clients;

3) Access a specific client, their profile, and their data submitted/their performance within the session via a number of graphic options (bar charts, graphs, numeric);

4) Make notes for each specific client after face-to-face sessions and about progress in between sessions through app data;

5) Rate each overall session;

6) Therapists have access to the daily triggers, achievements, outcomes and maladaptive behavior flagged notifications;

7) Directly contact the client/user, if deemed necessary; and

8) Therapists receive a notification that alerts them to their client's clinical decompensation or risky behaviors (more immediate than an on-call phone line).

Potential applications of this platform (i.e., samples of maladaptive behaviors) include: Addiction (substances of abuse/dependency); Anger management/domestic violence/intimate partner violence; Co-occurring maladaptive behaviors (e.g., addiction+domestic violence behaviors); Phobias; Trauma/PTSD; Depression; Anxiety disorders; Obsessive-Compulsive Disorder; Eating disorders; Smoking cessation; Weight control; and Compliance with medication (e.g., diabetes).

The disclosure uses an Avatar health coach and an interactive therapy platform of evidenced based cognitive behavioral therapy to target & treat maladaptive behaviors to improve treatment outcomes. This is a digital and interactive behavioral health coping skills platform that is grounded in science. It uses an Avatar as a Behavioral Health Therapy Coach. This is an adjunctive tool to any computerized or manually based Cognitive Behavioral Therapy Approach.

Cognitive Behavioral Therapy has been shown through numerous trials to be an effective standardized therapy that can be used to treat a range of behavioral health disorders. Practicing specific coping skill sets has been deemed necessary for health behavior change.

Cognitive Behavioral Therapy has a Coping Skills Assignment and the present disclosure developed a digital behavioral health platform for the coping skills assignment in place of the dogmatic paper and pencil approach. The digital platform is standardized and specific to target specific maladaptive behaviors. In this patient population (substance using offenders of intimate partner violence) have two maladaptive behaviors being targeted in an integrated manner as if both behaviors are inter-related and can trigger the other in negative ways. That is, anger/hostility and aggression can lead to substance use, misuse or relapse. Likewise, substance use can lead to impulsivity, disinhibition and aggressive behavior. Both commonly co-occur and should be treated in an integrated approach, especially when they do co-occur.

Practice exercises are designed to target the maladaptive behaviors. The Avatar Coach guides the client and interacts with the client while tracking progress.

In an embodiment, RITch and RITchie are named as an adult and juvenile Avatar Coach. The name “RITa” is used for a female Behavioral Therapy coach. Content of the Digital Coping Skill set is specific to the maladaptive behavior. Again, the following two maladaptive behaviors have been used w/clients as an example to show capabilities and effectiveness: Addiction and Intimate Partner Violence (e.g., aggression). The platform can be administered on a smart phone application, ipads/tablets or web-based.

The mechanisms believed to be important to implement positive, healthier, more prosocial behaviors are based on three unique components based in science and theory. The science driven mechanisms of change involved the following:

This platform uses 3 core components crucial to the ‘change mechanism” 1) social learning theory, 2) classical conditioning, and 3) operant conditioning are utilized at specific time points for targeting specific maladaptive behaviors within a digital platform-smart phone app, ipads, web-based, tablets. The platform is prescriptive, directive and structured. An Avatar Role Model for Patients signifies how social learning theory can be used to change behavior as this approach uses an Avatar as a Coach to show healthy behaviors.

Social learning theory posits that an individual learns a behavior by modeling the behavior that they see. The built in Avatar coach is a novel and innovative use of a “health role model” showing, shaping and guiding patients to practice and role model the healthier behaviors. The Behavioral Health Avatar is built based on science indicating that clients have a healthy positive interaction and alliance with an individual/therapist and/or coach that is genuine, empathic, rolls with resistance and avoids confrontation—a humanistic approach to motivate clients to change their behavior.

Classical conditioning is at play by assessing for triggers w/slider scales to understand use and trying to break the maladaptive associations. For example, this digital platform uses the Avatar as the Coach/Guide to help clients move the slider scale to disclose what their triggers are. That is, does family discord, anger/emotion, boredom, stress/financial problems, money, or positive emotions contribute to their substance use? The client is asked to slide the slider over to one or another side of the spectrum indicating whether they these stressors are ‘not true, somewhat true or often true’ for them. The categories are linked to a likert scale number so these numbers can be transcribed to a graph or other illustrative format to visually show clients if they are improving or not each week of therapy across the behavior areas being targeted (in this case it would be “Substance use Cravings, Substance Use, Anger, Conflicts, Aggressive Behaviors).

Operant conditioning is at play by rewarding and shaping the healthier behavior (e.g., the avatar coach uses verbal incentive and rewards such as “great job, way to go” and numerous affirmations. The interactive platform allows the clients ways of earning badges and awards for positive behavior change on an intermittent partial reward system). The intermittent partial reward system is also grounded in science. It is a schedule of reinforcement that has been shown in the literature to lead to the most sustained positive behavior change as the behavior is rewarded.

Additionally, this platform allows for patient engagement that is racially/gender specific. It is a digital platform w/an avatar that the patient chooses and customize (choice of race, gender, clothing etc.) to “personalize it.” This patient engagement is also linked to motivational enhancement strategies to move clients along to actually taking steps to change their behavior in healthier ways. By allowing patients to adapt this Avatar, it is a positive step they are attempting to make and invest in. It is utilizing cognitive dissonance, another form of science and theory to help motivate and change maladaptive behaviors. The Avatar is created to be likeable, positive and healthy therefore, the client attributes these characteristics to perception of self which leads to prosocial changes. That is, people tend to seek out consistency in their beliefs and perceptions.

This digital platform is standardized in an interactive fashion from Dr. Easton's behavioral health therapy manual and materials from an addiction and intimate partner violence treatment model.

Addiction and aggression are two maladaptive behaviors that are used as an example to show how this digital behavioral health platform and avatar coach can be effective and switched out with numerous other psychiatric disorders, problem behaviors and/or medical problems. Specific coping skills are taught via the digital platform. There are 12 topics to choose from in a menu of options. Clients practice with slider scales to answer the extent they are having cravings, practicing their skills and/or anger, aggression and conflict assessments. The sliders show an icon the patient can slide w/their finger on a smart phone app or digital tablet or click w/the mouse for a web-based application. The sliders are the following: “Never, Sometimes and Always” which align with a likert scale number. The program tracks their behavioral outcomes daily or weekly by having bar charts and visual representations that can be utilized by the patient, researchers and/or clinicians. This visual bar chart/graphics makes tracking easy. This is dynamic and uses behavioral science to increase positive behavior change through “practicing by using this digital platform and its interactive capabilities.” Again, this platform is standardized and you can switch out any “maladaptive behavior (e.g., addiction, aggression, anger, eating disorders, smoking, depression, anxiety disorders/phobias/trauma, psychosis, OCD, ADHD, conduct disorders, personality disorders, addictive disorders, and medical related problems). The patient population can be personalized by age (adult, juvenile, senior) gender and race. Content is specific to the maladaptive behavior, such as, addiction and aggression.

There are 12 sessions with specific therapy content to be used in this interactive, digital behavioral health platform. See the Session Numbers/Topics, Structure and Format and Content of the Skills set Administered.

Total Number of Skill Sets to Learn Via the Program in Order. The Assessment will suggest which combination is suggested based on the User's input. Each interaction/each day or week, the program keeps track and continuously modifies which skill sets are suggested based on their suggests and integrates assessment responses, skills used and behavioral progress.

Behavioral Targets: Addiction and Aggressive Behavior

Skills Topic 1: Introduction

Skills Topic 2: Identifying Triggers for Substance Use and Triggers for Anger/Conflict

Skills Topic 3: Awareness of Anger

Skills Topic 4: Managing Anger

Skills Topic 5: Coping With Criticism

Skills Topic 6: Communication Skills Training (Non-verbal Module)

Skills Topic 7: Communication Skills Training (Verbal Module)

Skills Topic 8: Communication Skills Training (Effects on Family/Children)

Skills Topic 9: Assertiveness NOT Aggression

Skills Topic 10: Managing Negative Moods and Depression

Skills Topic 11: Problem Solving

Skills Topic 12: Preparing for Emergencies

This Digital Interactive CBT coping skill exercise could be used across numerous psychiatric and medical disorders. It consists of changing the maladaptive/unhealthy behavior that individuals, doctors or clinicians are attempting to change.

This could be used across All Addictive Disorders, Anxiety Disorders (Trauma, PTSD, Phobias, Generalized Anxiety, OCD), Depression, Bipolar Disorder, Eating Disorders, Psychotic Disorders, Conduct Disorder, ADHD. The platform will allow the content to be changed so that it targets the specific Disorder and its relevant maladaptive behavior. This is consistent with Cognitive Behavioral Therapy, flexible, prescriptive, standardized and active.

Moreover, this can be adapted and used with male and female clients, with Adult populations, juvenile, children and elder populations. It can be adapted to race and sexual orientation.

This is a Digital, Interactive Behavioral Health Therapy Platform for CBT Coping Skill Exercises with a Built in Personalized Avatar Coach w/Built in Rewards. The present invention attempts to use all that technology has to offer to combine key science and theory in the “behavioral therapy” venue in a digitized fashion w/avatars, rewards and the possible ingredients of science and theory that leads to change (social learning theory, operant conditioning/intermittent partial reward system, classical conditioning w/the Avatar who is built to be humanistic/genuine).

The invention can be standardized, personalized, client centered, targets specific maladaptive behaviors, using a number of science and theories as important ingredients that lead to changing maladaptive behaviors into adaptive/prosocial behaviors. It is digitized, easily accessible, inexpensive/cost effective, fun and easily disseminated and sustainable. Moreover, maladaptive behaviors tend to co-occur so this innovative approach allows for integration of care in comprehensive, standardized and integrated manner.

Potential Commercial Uses include the following Target Customers:
Clinicians and medical professionals working with patients that have behavioral health disorders (primary or secondary)
Patients wanting to change a negative and unhealthy behavior (Hospital Settings, Urgent Care, Outpatient Clinics)
Clinicians and medical professionals who treat clients with addiction and intimate partner violence
Researchers wanting to study behavioral health disorders
Clinical private practices and organizations that focus on behavioral treatments
Hospital Settings that treat psychiatric disorders
Criminal Justice Systems that have diversionary treatment programs (mental health court, drug court, intimate partner violence courts, veterans' courts.

Any medical or behavioral health disorder can be substituted into this platform. The Cognitive Behavioral Therapy Content can be altered to target a specific maladaptive behavior needing to be changed.

The disclosure will be further illustrated with reference to the following specific examples. It is understood that these examples are given by way of illustration and are not meant to limit the disclosure or the claims to follow.

### Example 1

John Doe is evaluated for treatment at Catholic Family Services following an arrest for disorderly conduct. He was drinking, screaming and yelling at his partner.

After John Doe was accepted into treatment and assigned to a Cognitive Behavioral Therapy Group or Cognitive Behavioral individual (1:1) therapy with his clinician, he met with his therapist.

His therapist gave him a tablet to practice dCBT (digitized, Avatar Assisted Cognitive Behavioral Therapy) program. The therapist followed along on her iPad to show John how to use the platform.

John turned on the device and clicked on the dCBT button and entered his login information (username and password). After he entered the login information, an Avatar enters the screen and thanks the user by their first name based on the first assessment question (“Thank you John for interacting with me”). The Avatar congratulates the user for interacting and there is a pleasant sound and a color burst as the Avatar thanks the user (Great job, John ! Thank you for taking the first step toward healthy behavior change”—a soft chime sound and a pleasant burst of a stars enters the screen). The Avatar then goes through a tutorial which offers the user a menu of options regarding customizing their Avatar to be their personalized Behavioral Therapy Coach. The Avatar asks the user (John) to personalize their Coach by choosing gender (male, female, non-gender specific option), ethnicity/racial option (choose skin colors—light complexion, light brown, brown, dark brown, very dark brown), hair style (long hair, short hair, short spikey hair, brush cut, short curly hair), clothing options (female khaki slacks, female dress jeans, female dress shirt, female casual shirt, dress with and without scarf, male khaki slacks, male dress jeans, male dress shirt, male casual shirt, male dress shirt with or without a tie, non-gender specific khaki slacks, dress jeans, dress shirt, casual shirt, dress, scarves, ties) and environment (Avatar Coach w/An Office Background or Avatar Coach with Nature Scene in background).

Once the choices have been made, the Avatar comes on the Screen with the gender, race, hair, clothing and environmental choices integrated into their Avatar of choice. The Avatar then thanks John for creating him (“Thank you, John for taking the time to create me. I am your personalized Behavioral Therapy Coach”) and pleasant sound occurs (soft ping) w/a color burst.

The Avatar then asks John to take a short assessment and asks him questions about his age, race, relationship status, relationship satisfaction, employment status, educational status, legal status, psychiatric symptoms (brief symptom checklist-depression, medical status, anxiety, anger, substance use, conflict levels at home, conflict levels at work etc., primary drug of choice, secondary drug of choice, person to whom conflict is with). The questions are posed by the Avatar to the user (John). The user uses a slider scale and slides their finger along a scale that is linked to a likert scale of numbers. The scale notes whether they are experiencing any problems ranging from no problems (score=0) toward the other end of the scale, which is always experiencing the problem behavior (score=10).

In addition to the overall baseline profile, each interaction begins assessing the following behaviors pre-interaction:

On a likert scale from 0 (no problems) to 10 (always experiencing the problem)

Craving for substances;

Use of Substance;

Anger,

Conflict Level,

Aggression,

Promises kept,

Out of Session Homework Exercises Practiced

Each of these Problem Behaviors and Level of Practice Done is tracked with each interaction across 12 weeks.

Based on the functional analysis and the assessment questions, the program recommends a menu of skill sets based on the problem behavior ratings. The user chooses with one or which combination to practice.

For this purpose, Triggers and Anger Management are suggested and the User will interact w/the Avatar to learn about them and to apply them.

“Name”: Identifying Triggers,
“Description”: “this is the description for the dashboard”,
“Narration”: [

“Hi Thank you again for interacting. It's time to get started,

It is important to start to recognize some of your triggers in your life. That is, triggers to use and/or triggers to lose control of your emotions and/or temper

On my left I've listed some common triggers for substance use. I would appreciate it if you could scale each trigger based on how much they tempt you to use your substance of choice, with zero meaning never and 10 meaning always

```
],
“Sliders”: [
    “Friends,
    “Family”,
    “Emotion such as anger,
    “Anxiety/worry,
    “Excess cash/money,
    “Boredom,
    “Conflicts with you intimate partner (spouse, girlfriend, boyfriend),
    “Conflicts with others (family, friends, co-workers),
    “Work problems
],
“Narration”: [
```

“Next I would like you to consider some of your triggers for aggression. Again, people, places, situations and emotions can all trigger an urge to lose control of your temper, which in turn, can lead to an aggressive behavior. What are your triggers

```
],
“Sliders”: [
    “Substance use,
    “Anger”,
    “Sadness,
    “Anxiety/worry,
    “Money problems,
    “Depression,
    “Work problems,
    “Conflicts with intimate partner,
    “Conflicts with others (family, friends, co-worker)
],
Narration”: [
```

“Please scale the items on my left based on the frequency in which they occur. I would like you to consider the interaction between your substance abuse and aggressive behaviors?

],
“Sliders”: [

“To what degree do you have arguments (yelling/screaming/name calling) while using,

“To what degree do you have arguments (yelling/screaming/name calling) while sober”,

“To what degree do you use physical aggression while using,

“To what degree do you use physical aggression while sober,

“To what degree do you resolve conflicts while using,

“To what degree do you resolve conflicts while sober

],
Narration”: [

“Please scale the items on my left based on the frequency in which they occur. Thank you very much for sharing your triggers with me! It really means a lot that you're taking the time and action steps to improve your health because you are worth it!,

Remember that avoiding triggers is the most helpful step you can take, but we both know that is not always easy or possible to avoid things. So, please consider using some coping skills that can help you get through some of those challenging situations,

I Coping skills can be any healthy activity that distracts yourself from thinking about your triggers. They help keep you busy when you feel a trigger is coming on. It keeps your thoughts and feelings away from aggression and substance abuse. Some healthy examples of distracting activities involve the following

],
“Sliders”: [
going for a walk or a hike,
going fishing,
listening to music,
drawing a picture,
playing a video games,
exercising,
going to the gym,
slowly counting to 10,
mindfulness and/or meditating
],
Narration”: [

“Can you rate from 0-10 to what degree you are most likely to do any of the coping skills listed above (10 being most likely)i

Now I would like you to practice one right now. If you are alone, practice this right now, if you are not alone, merely think it to yourself quietly. Stand still or sit down and remain calm. Count slowly from 1-10

Now I would like you to try this meditation activity (imagine yourself on a beach or some place you would love to be, breath slowly. Imagine it is warm and nice out. It is peaceful. imagine slowly drawing the number 1 in the sand or in the air, erase it with your hand, then draw the number two, erase it and keep repeating this, drawing, 3, 4, 5, 6, 7, 8, 9 until you get up to the number 10). Drawing slowly, erasing slowly each number. Take a deep breath. You did it.

],
“Sliders”: [

To what degree did you actually try this meditation activity, with 10 meaning you tried it all,

To what degree di you find it helpful, with 10 meaning that you found it very helpful

],
“Narration”: [

Please try these anytime you feel an urge to use or an urge to lose control of your temper. Again, if you are not able to control your temper, the Time out activity can be used to avoid an emergency

Narration: [Great Work. I look forward to interacting again. Before we end, let's practice the promises,

I can appreciate that you do not want to participate in any angry touching,

I can appreciate that you do not want to participate in any in any yelling, screaming or name-calling,

I can appreciate that you do not want to participate in any threatening or harassing behaviors,

I can appreciate that you want to try your best to reduce your substance use,

And finally, it is important to remember that a slip to substance use doesn't have to mean a slip to aggression!,

Thank you again for your time today, you really did a great job.

```
],
“Quiz”: [
    {
      “Question”: “Cravings are NOT normal. They will not go away”,
      “Answers”: [ “True”, “False” ],
      “Correct” : “False, they are normal and they will go away”,
“Question”: “Substance use can lead to aggression”,
      “Answers”: [ “True”, “False” ],
      “Correct” : “True, substance use can facilitate aggression”,
    }
    ]
  }
  ]
}
“Name”: Awareness of Anger,
“Description”: “this is the description for the dashboard”,
“Narration”: [
```

Hello. It's great to see you interacting again. Today we are going to learn about anger. We are especially interested in how you manage your anger around your intimate partner,

Again, it is important to know that anger is actually a normal emotion. There is a big difference between anger and aggression. Anger is a feeling. How you handle your anger can determine whether it is healthy or not healthy. If your anger turns into a behavior intended to injure or hurt your intimate partner, then it becomes aggressive. Aggression is not healthy and can cause numerous negative consequences. Being aware of your anger can help you choose how to manage it,

For example, There are two types of anger known as Direct and Indirect that you may experience from various triggers,

Some examples of direct anger include,

Being bullied, told what to do, or bossed around,

Being pushed, shoved or hi,

Being given the finger or other rude gestures,

Being treated unfairly,

Getting frustrated at somebody because you can't accomplish your goals,

Examples of indirect anger include feeling that you are being blamed, thinking that someone is disapproving of you, or feeling that too many demands are being made of you,

Can you tell me how often you experience direct anger in your life?

],
“Sliders”: [
Being bullied, told what to do, or bossed around,
You were pushed, shoved or hit,
Someone is giving you the finger or other rude gestures,
Feeling that you are being treated unfairly,
Getting frustrated at somebody because you cannot accomplish your
],
Narration: [

goals

Can you tell me how often you experience indirect anger in your life and especially indirect anger you experience with your partner? i

],
“Sliders”: [

Feeling that you are being blamed,

Thinking that someone is disapproving of you,

Feeling that too many demands are being made of you.

],
Narration: [

Ok thank you for sharing this with me. Next I would like to discuss some signals that your body is telling you that you are feeling angry. When you become angry how often do the following physical signs occur?

],
“Sliders”: [
Your face gets hot and red,
Your jaw gets tense and stiff,
You start pacing and fidgeting,
You start sweating,
You get loud and yell,
Your heart rate increases
],
Narration: [

Thank you for your feedback. Let's remember that these physical symptoms are WARNING SIGNALs you get from your body. Your body is giving you some warning signs so you can intervene, stop and channel your anger in healthier ways and not get aggressive.

Also, there are many signs we are given based on what thoughts we are experiencing. Negative thoughts regarding often accompany the physical signs. How often do these occur?

],
“Sliders”: [
Feeling like things are out of control,
Cursing or swearing in your head,
Imagining physical harm to others,
Narration: [

Thank you for sharing this. Let's remember the warning signal you are getting from your thoughts.

Many emotions often occur along with anger, which emotions do you feel when you are angry?i

],
“Sliders”: [
Frustrated,
Anxious,
Blue/Depressed,
Wound up,
Narration: [

Thank you! Let's remember that top emotional state that accompanies anger.

Several unpleasant behaviors often accompany anger. How often do these behaviors occur when you are angry?

],
“Sliders”: [
Sleep Problems,
Storming out of rooms when in conflict with others,
Getting into fights with you intimate partner],
Narration: [

Again, thank you for your answers. I only have a few more questions related to how you deal with your anger,

I am going to discuss three unhealthy ways in which people often deal with their anger. It is not uncommon for people to report that they are Exploders and Stuffers,

Exploders are likely to act impulsively when angry. For example an exploder may get angry and instantly start hitting things before thinking about it.

],
“Sliders”: [

How often do you feel like you behave like an exploder in your intimate partner relationships?

],Narration: [

Stuffers let their anger build up until it explodes on them like a kettle boiling on the stove until the steam just blows the lid off]

“Sliders”: [

How often do you feel like you behave like a stuffer? That is, you stuff your anger repeatedly until you just lose your temper? î]

Narration: [

Ok great, thank you very much for talking with me,

There are several healthy coping skills that I can give you that will help you cope with your anger. When you begin to notice the signs and symptoms of anger you should try to use these techniques to calm yourself down,

Give yourself a time out phrase like chill out or calm down,

Count to ten inside your head and breathe,

Drink a cool glass of water

Take a moment to sit and relax,

Go for a walk,

Which one works best for you? Preform this activity that occupies your time and distracts you from the source of your anger.

Let's try counting to 10 right now. Take a few seconds to count slowly to 10,

Ok great thank you very much for talking with me!

],
“Sliders”: [
To what degree did you actually try this activity?,
To what degree did you find it helpful?],
“Narration”: [

All of these anger coping skill exercises help prevent anger from turning into aggression.

```
],
“Quiz”: [
    {
    “Question”: “Anger is a feeling. Anger is always unhealthy”,
    “Answers”: [ “True”, “False” ],
    “Correct” : “ False, anger is a normal emotion. It is only when it
```

becomes aggressive and causes injury that it becomes unhealthy”,

```
    “Question”: “Indirect anger is  ”,
    “Answers”: [ “a. feeling blamed and feeling too many demands”,
```

“b. thinking someone is disapproving of you”, c. being bullied, a & b, e. b & ci],

```
    “Correct” : “d. a & b”
    “Question”: “Direct anger is  ”,
    “Answers”: [ “a. being bullied”, “b. being pushed or shoved”, c.
```

being blamed, d. a & c, e. a & b],

```
    “Correct” : “e. a & b”
    }
  ]
  }
]
```

The program concludes the first session by the Avatar therapy coach thanking John. “Thank you, John for all your hard work and steps at improving your health. Today you completed ______% of the skill sets recommended. You scored ______ on your quiz and you achieved ______ badges.

You are making great progress.

During each of the slider interactions, there are sounds to indicate a choice is made. During the positive affirmations (great job, way to go, you are making great progress), positive sounds and light and bursts of color occur (subtle but pleasant and rewarding).

The program keeps track of how the user responded, questions answered and behaviors that are improving or not. Suggested skill sets linked to positive behavior change are being stored and re-assessed and integrated into the algorithm.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed:

1. A method for administering a digital behavioral health platform to an individual, comprising:
customizing a digital avatar health coach;
practicing a set of coping skills provided by an interactive therapy platform of evidenced based cognitive behavioral therapy which targets and treats maladaptive behaviors in the individual to improve treatment outcomes; and
interacting with the avatar health coach to provide personalized feedback, wherein the digital platform is standardized and specific to target specific maladaptive behaviors and implements positive, healthier, and prosocial behaviors which incorporates the mechanisms of social learning theory, classical conditioning, and operant conditioning based on the feedback.

2. A digital behavioral health platform for an individual, comprising:
a customizable digital avatar health coach; and
an interactive therapy platform of evidenced based cognitive behavioral therapy which targets and treats maladaptive behaviors in the individual to improve treatment outcomes based on the feedback from practicing a set of coping skills.
